(19) United States
(12) Patent Application Publication
(10) Pub. No.: US 2024/0161288 A1
(43) Pub. Date: May 16, 2024

(54) ENDOSCOPE SYSTEM, OPERATION METHOD OF ENDOSCOPE SYSTEM, AND PROCESSOR

(71) Applicant: FUJIFILM Corporation, Tokyo (JP)

(72) Inventor: Hisashi ENDO, Kanagawa (JP)

(73) Assignee: FUJIFILM Corporation, Tokyo (JP)

(21) Appl. No.: 18/502,065

(22) Filed: Nov. 5, 2023

(30) Foreign Application Priority Data
Nov. 11, 2022 (JP) 2022-181078

(51) Int. Cl. G06T 7/00; A61B 1/00

(52) U.S. Cl. CPC G06T 7/0012; A61B 1/000094; A61B 1/00045; G06T 2207/10068; G06T 2207/30096; G06T 2207/30168

(57) ABSTRACT

An endoscope system includes an endoscope and a processor device. The processor device functions as a lesion detection unit and an abnormality detection unit. The lesion detection unit analyzes an image captured by the endoscope to detect a lesion part and displays a detection result on a display. The abnormality detection unit analyzes the image captured by the endoscope to detect an abnormality, and stores the image in which the abnormality has been detected in a final storage unit after an endoscopic examination end.

### CROSS-REFERENCE TO RELATED APPLICATIONS

This application claims priority under 35 U.S.C. § 119(a) to Japanese Patent Application No. 2022-181078 filed on 11 Nov. 2022. The above application is hereby expressly incorporated by reference, in its entirety, into the present application.

### BACKGROUND OF THE INVENTION

### 1. Field of the Invention

The present invention relates to an endoscope system provided with an endoscope and a processor, an operation method of an endoscope system, and a processor.

### 2. Description of the Related Art

In an endoscope system including an endoscope and a processor device (processor), or an image processing processor device (processor) connected to the endoscope, an examination (endoscopic examination) is performed using an image (endoscopic image) captured by the endoscope. It is preferable that the endoscopic image is obtained in a state in which the endoscope and the imaging environment are in a good condition, but in a case where the endoscope is malfunctioning or in a case where the imaging environment is not in a good condition due to factors such as dirt attachment, abnormalities may occur in the endoscopic image. In a case where such an abnormality occurs, it is necessary to perform repair or maintenance.

JP2019-187494A (corresponding to US2019/320880A1) describes a configuration in which an endoscopic image is analyzed, an abnormality is detected, and the server is notified. In this way, by issuing notification of the abnormality, maintenance can be performed at an appropriate timing.

### SUMMARY OF THE INVENTION

However, JP2019-187494A has a problem in that a processing load during an endoscopic examination is significant. That is, in a case where an abnormality is detected as in JP2019-187494A, processing of not only detecting the abnormality but also storing the endoscopic image in which the abnormality has been detected for storage (storing the endoscopic image in a final storage device for storage (first storage device)) for purposes such as investigating causes is performed. In addition, in the endoscopic examination, processing of analyzing the endoscopic image to detect a lesion part, output a detection result (display the detection result on a monitor), or the like is also performed. Performing all of the processing during the endoscopic examination has a significant processing load.

The present invention has been made in view of the above background, and an object of the present invention is to provide an endoscope system, an operation method of an endoscope system, and a processor capable of reducing a processing load during an endoscopic examination.

In order to achieve the above object, according to an aspect of the present invention, there is provided an endoscope system comprising: an endoscope that images an inside of a body cavity; and a processor, in which the processor is configured to: detect a lesion part from an endoscopic image captured by the endoscope, and display a detection result of the lesion part on a monitor during an endoscopic examination using the endoscope; and detect an abnormality that has occurred in the endoscopic image, and store the endoscopic image in which the abnormality has been detected in a first storage device for storage after an end of the endoscopic examination.

The processor may be configured to store the endoscopic image in a second storage device for temporary storage during the endoscopic examination, and detect the abnormality using the endoscopic image stored in the second storage device after the end of the endoscopic examination.

The processor may be configured to detect the abnormality during the endoscopic examination.

It is preferable that the processor is configured to: in a case of detecting the abnormality during the endoscopic examination, detect the abnormality before detecting the lesion part; and exclude the endoscopic image in which the abnormality has been detected from a detection target of the lesion part.

It is preferable that the processor is configured to detect the lesion part on a thinned-out endoscopic image which is a part of a plurality of the endoscopic images.

It is preferable that the processor is configured to store the endoscopic image in which the abnormality has been detected in the first storage device in a form accessible to an external device.

It is preferable that the endoscopic image includes personal information including at least any of subject person information regarding a subject person of the endoscopic examination or operator information regarding an operator of the endoscopic examination, and that the processor is configured to erase the personal information from the endoscopic image in which the abnormality has been detected and store the endoscopic image in the first storage device.

It is preferable that the processor is configured to set an allowable access range for the endoscopic image in which the abnormality has been detected to a range different from an allowable access range for an endoscopic image used for the endoscopic examination.

It is preferable that the processor is configured to set an allowable editing type for the endoscopic image in which the abnormality has been detected to a type different from an allowable editing type for an endoscopic image used for the endoscopic examination.

It is preferable that the abnormality includes an abnormality based on an internal factor including a malfunction occurring in a main body of the endoscope and an abnormality based on an external factor occurring outside the main body of the endoscope, and that the processor is configured to detect the abnormality based on the internal factor.

In addition, in order to achieve the above object, according to another aspect of the present invention, there is provided an operation method of an endoscope system, comprising: a step of detecting a lesion part from an endoscopic image captured by an endoscope, and displaying a detection result of the lesion part on a monitor during an endoscopic examination using the endoscope; and a step of detecting an abnormality that has occurred in the endoscopic image, and storing the endoscopic image in which the abnormality has been detected in a first storage device for storage after an end of the endoscopic examination.

Further, in order to achieve the above object, according to still another aspect of the present invention, there is provided a processor that is connected to an endoscope system including an endoscope which images an inside of a body cavity and that receives an input of an endoscopic image captured by the endoscope during an endoscopic examination using the endoscope, the processor configured to: during the endoscopic examination, detect a lesion part from the endoscopic image and input a detection result of the lesion part to the endoscope system; and detect an abnormality that has occurred in the endoscopic image, and store the endoscopic image in which the abnormality has been detected in a first storage device for storage after an end of the endoscopic examination.

According to the present invention, a processing load during the endoscopic examination can be reduced.

### BRIEF DESCRIPTION OF THE DRAWINGS

FIG. 1 is a block diagram showing a configuration of an endoscope system.

FIG. 2 is a block diagram showing a configuration and a function of a processor device.

FIG. 3 is an explanatory diagram showing a flow of processing of the processor device.

FIG. 4 is a flowchart showing the flow of the processing of the processor device.

FIG. 5 is an explanatory diagram showing a flow of another processing of the processor device.

FIG. 6 is a flowchart showing the flow of the processing of the processor device.

FIG. 7 is a block diagram showing a configuration and a function of an image processing processor device.

### DESCRIPTION OF THE PREFERRED EMBODIMENTS

### First Embodiment

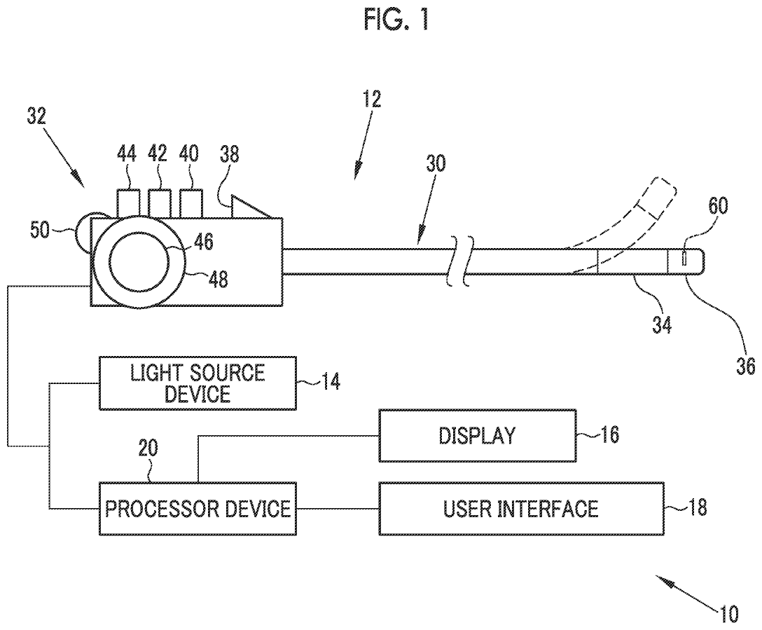

As shown in FIG. 1, an endoscope system 10 of a first embodiment includes an endoscope 12, a light source device 14, a display 16 (monitor), a user interface 18, and a processor device 20 (processor).

In the endoscope system 10, the endoscope 12 is optically connected to the light source device 14 and is electrically connected to the processor device 20. Additionally, in the endoscope system 10, the processor device 20 is electrically connected to each unit (the endoscope 12, the light source device 14, the display 16, and the user interface 18) of the endoscope system 10.

The endoscope 12 includes an insertion part 30 to be inserted into a body (body cavity) of an observation target, an operation part 32 connected to a proximal end side of the insertion part 30, and a bending portion 34 and a distal end portion 36 that are provided on a distal end side of the insertion part 30. The operation part 32 is provided with an inlet 38 of a forceps channel for inserting a treatment tool, such as a forceps. In addition, the operation part 32 is provided with various operation members that receive operations from a user, such as bending of the bending portion 34, zooming during subject imaging, a still image/video capturing instruction, switching of an imaging mode, and air supply and water supply. In the present embodiment, as the above-described operation members, rotating dials 40, 42, and 44 that are rotated for operations and press buttons 46, 48, and 50 that are pressed for operations are provided.

The distal end portion 36 is provided with an illumination window for emitting illumination light, an observation window for receiving reflected light reflected by a subject after the light emission from the illumination window, an outlet of the forceps channel, an air and water supply port, and the like. In addition, the distal end portion 36 includes an image sensor 60 provided behind the observation window. The image sensor 60 is an image sensor that outputs a captured image as a digital image signal, such as a charge coupled device (CCD) or a complementary MOS (CMOS). The image (endoscopic image) captured by the image sensor 60 is input to the processor device 20.

The light source device 14 supplies illumination light to the endoscope 12. The display 16 is, for example, a well-known liquid crystal display and displays the image captured by the endoscope 12 (image sensor 60), a detection result in a case where a lesion part, which will be described below, is detected, and the like. The user interface 18 is an input device for performing an input to the processor device 20 and the like and is, for example, a keyboard, a mouse, a foot pedal, a touch panel, a microphone, and/or a motion sensor.

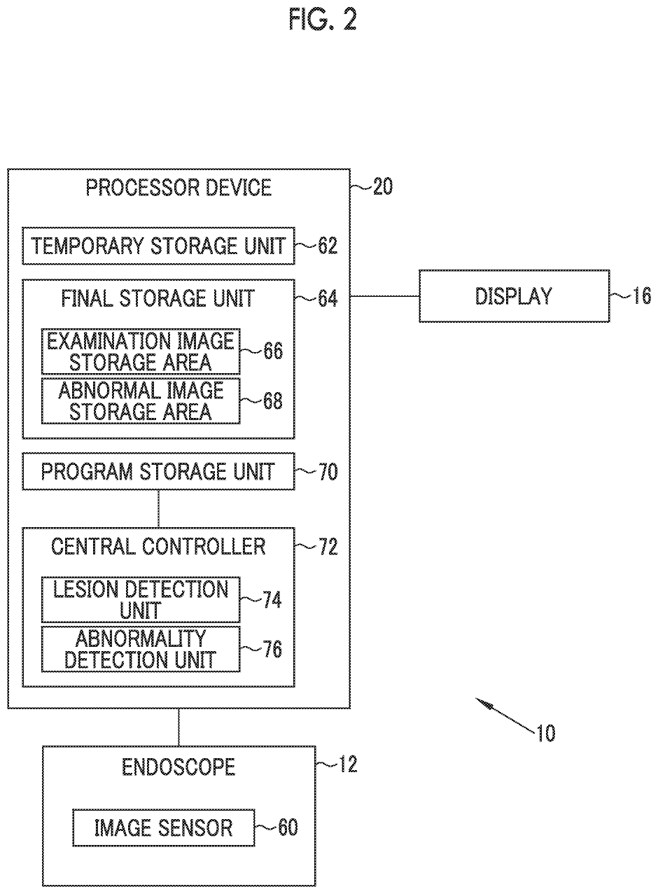

As shown in FIG. 2, the processor device 20 is provided with a temporary storage unit 62 (second storage device) and a final storage unit 64 (first storage device). Although the temporary storage unit 62 and the final storage unit 64 both store the image captured by the endoscope 12 (image sensor 60), the temporary storage unit 62 is used for temporary storage of information such as images, while the final storage unit 64 is used for storage (long-term storage) of information such as images.

The final storage unit 64 is provided with an examination image storage area 66 and an abnormal image storage area 68. In a case where an instruction to capture a still image/video is issued during the endoscopic examination using the endoscope 12, the still image/video captured based on this instruction is stored in the examination image storage area 66. In addition, an image showing the detection result of the lesion part obtained by a lesion detection unit 74, which will be described below, or a recorded image of a display screen of the display 16 is also stored in the examination image storage area 66. Meanwhile, an image in which an abnormality has been detected by an abnormality detection unit 76, which will be described below, is stored in the abnormal image storage area 68.

The final storage unit 64 is provided in an accessible manner to an external device (for example, a terminal of a doctor or other personnel of a medical institution in which the endoscope 12 is disposed, a terminal of repair personnel of the manufacturer of the endoscope 12, or the like) (not shown) connected to the processor device 20 via a known network (not shown) such as a local area network (LAN) or the Internet. By accessing the final storage unit 64 from such an external device, it is possible to view and manage the stored information.

The processor device 20 is provided with a program storage unit 70 and a central controller 72 in addition to the temporary storage unit 62 and the final storage unit 64 described above. A program related to various types of processing, control, or the like is stored in the program storage unit 70. The central controller 72 functions as the lesion detection unit 74 and the abnormality detection unit 76 by operating the program stored in the program storage unit 70.

The lesion detection unit 74 detects the lesion part by analyzing the image captured by the endoscope 12 (image sensor 60) and displays the detection result on the display 16. The lesion part is, for example, a tumor part, an inflammatory part (including a portion with a change such as bleeding or atrophy in addition to a so-called inflammation), a colonic diverticulum, a treatment scar (an endoscopic mucosal resection (EMR) scar, an endoscopic submucosal dissection (ESD) scar, or a clip portion), a bleeding point, a perforation, a vascular abnormality, a cauterization scar due to heating or a marking part marked by coloring with a coloring agent, a fluorescent agent, or the like, or a biopsy part subjected to a biological examination (so-called biopsy).

The display of the detection result of the lesion part is performed, for example, by generating a lesion-enhanced image in which the lesion part is enhanced and displaying the image on the display 16. The lesion-enhanced image is an image (superimposed image) in which information indicating the lesion part (an image with the lesion part colored, a contour line of the lesion part, a marker surrounding the lesion part, an indicator such as an arrow pointing to the lesion part, or the like) is superimposed on an image (original image) of a detection source of the lesion part, or an image (switching image) in which the original image and the superimposed image are temporally switched. Of course, the detection result of the lesion part may be displayed by displaying the lesion-enhanced image (the superimposed image or the switching image) alongside the original image. In addition, the detection result of the lesion part may be displayed by displaying text information indicating the position or presence of the lesion part on the display 16.

The lesion detection unit 74 may detect the lesion part for all the images captured by the endoscope 12. However, since several tens of images are captured per second during the endoscopic examination, detecting the lesion part for all the images has a significant processing load. Therefore, in the present embodiment, the lesion detection unit 74 is configured to detect the lesion part for a thinned-out image obtained through thinning out, which is a part of the images captured by the endoscope 12. Specifically, for example, a configuration is employed in which the lesion part is detected once every 0.2 seconds (for five images per second) or the lesion part is detected for every one image out of five input images (every fourth image). By doing so, the processing load during the endoscopic examination can be reduced.

The abnormality detection unit 76 detects the abnormality by analyzing the image captured by the endoscope 12 (image sensor 60). The abnormality includes an abnormality based on an internal factor occurring in a main body of the endoscope 12 and an abnormality based on an external factor occurring outside the main body of the endoscope 12. The abnormality based on the internal factor is an abnormality caused by a malfunction of the endoscope and is, for example, a burn in the image sensor 60, a pixel defect, a breakage in a transmission path (a circuit or a cable) for image signals, and a scratch on an optical path (a scratch on the observation window or an imaging lens). Since the abnormality based on the internal factor cannot be resolved through normal maintenance (which can be performed at a medical institution in which the endoscope 12 is disposed) such as cleaning, more advanced maintenance is required such as repair performed by the manufacturer of the endoscope 12. On the other hand, the abnormality based on the external factor is caused by, for example, dirt (body fluids, residue, or the like) attachment to the observation window, and can be resolved through normal maintenance.

As described above, since the abnormality includes the abnormality based on the internal factor and the abnormality based on the external factor, both of these abnormalities may be detected by the abnormality detection unit 76. However, in a case where both the abnormalities are detected, for example, there is a problem of excessive response to the abnormalities, such as assuming that the endoscope 12 is malfunctioning even for the abnormality that can be resolved through normal maintenance and requesting repair from the manufacturer of the endoscope 12. Therefore, in the present embodiment, a configuration is employed in which only the abnormality based on the internal factor is detected. That is, the abnormality detection unit 76 of the present embodiment does not detect the abnormality that can be resolved through normal maintenance, but detects the abnormality that requires more advanced maintenance. By doing so, it is possible to prevent an excessive response to the abnormality.

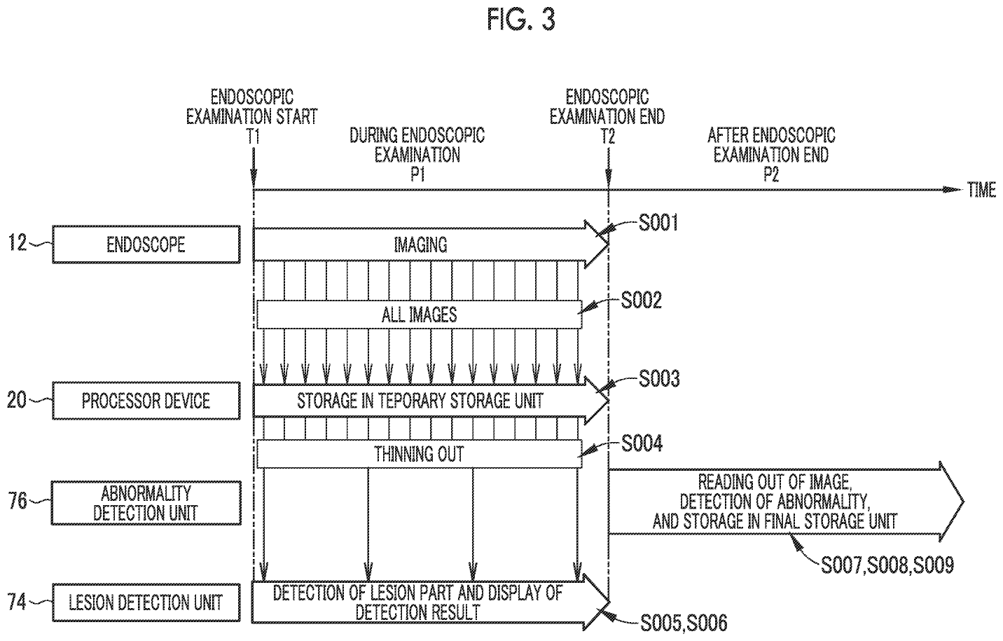

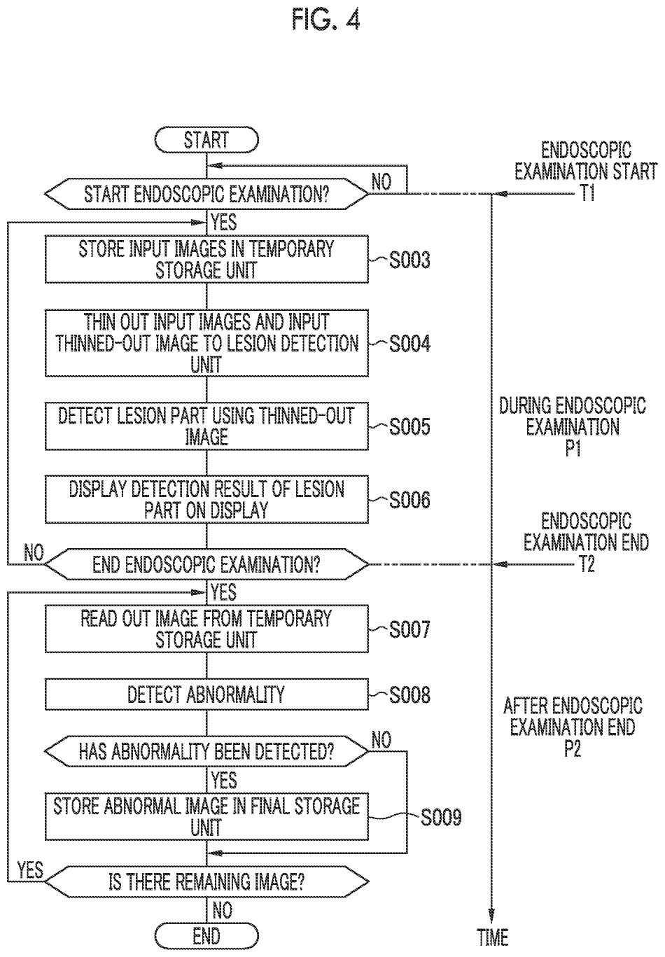

Hereinafter, a flow of processing of the processor device 20 will be described with reference to FIGS. 3 and 4. With the endoscopic examination start (T1), imaging with the endoscope 12 (image sensor 60) is performed (S001), and the captured image is input to the processor device 20 (S002). During the endoscopic examination (P1), imaging is performed at several tens of cycles per second (S001), and the captured images are sequentially input to the processor device 20 (S002).

During the endoscopic examination (P1), the processor device 20 stores all the input images in the temporary storage unit 62 (S003) and inputs a thinned-out image obtained through thinning out (S004), which is a part of the input images, to the lesion detection unit 74. The lesion detection unit 74 analyzes the input image to detect the lesion part (S005) and displays the detection result on the display 16 (S006). The storage of the image in the temporary storage unit 62 (S003), the detection of the lesion part by the lesion detection unit 74 (S005), and the display of the detection result (S006) are repeated during the endoscopic examination (P1), that is, from the endoscopic examination start (T1) (the input start of the image from the endoscope 12) to the endoscopic examination end (T2) (the input stop of the image from the endoscope 12).

In the present embodiment, the input stop of the image from the endoscope 12 is regarded as the endoscopic examination end (T2), but the present invention is not limited to this. For example, the detection of operations or processing performed at the end of the endoscopic examination, such as pressing an examination end button provided in the endoscope 12 or the processor device 20, may be regarded as the endoscopic examination end (T2).

With the endoscopic examination end (T2), that is, after the endoscopic examination end (P2), the processor device 20 operates the abnormality detection unit 76 to detect the abnormality. The abnormality detection unit 76 reads out the image stored in the temporary storage unit 62 (S007) and detects the abnormality using the read-out image (S008). Then, in a case where the abnormality is detected, the image in which the abnormality has been detected (hereinafter, referred to as an abnormal image) is stored in the abnormal image storage area 68 of the final storage unit 64 (S009). The detection of the abnormality is performed for all the images stored in the temporary storage unit 62. As described above, in the present embodiment, a configuration is employed in which the detection of the abnormality and the storage of the abnormal image in the final storage unit 64 (abnormal image storage area 68) are performed after the endoscopic examination end (P2), so that the processing load during the endoscopic examination (P1) can be reduced.

In addition to the abnormal image, information regarding the detected abnormality (which is information indicating a type of the abnormality and the like and hereinafter referred to as abnormality information) may be stored in the final storage unit 64 (abnormal image storage area 68) in association with the abnormal image. In this case, the abnormality information may be stored as accompanying information of the abnormal image in a form attached to the abnormal image. In addition, the abnormality information may be stored separately from the abnormal image. In this case, correspondence information indicating a correspondence relationship between the abnormal image and the abnormality information need only be stored by being attached to the abnormal image or the abnormality information, or need only be stored independently.

### Second Embodiment

In the above first embodiment, both the detection of the abnormality (S008) and the storage of the abnormal image in the final storage unit 64 (abnormal image storage area 68) (S009) are performed after the endoscopic examination end (P2), but in a second embodiment, the detection of the abnormality (S008) is performed during the endoscopic examination (P1), and the storage of the abnormal image in the final storage unit 64 (abnormal image storage area 68) (S009) is performed after the endoscopic examination end (P2). In the following description, the same members as those in the above-described first embodiment are designated by the same reference numerals, and the description thereof will be omitted.

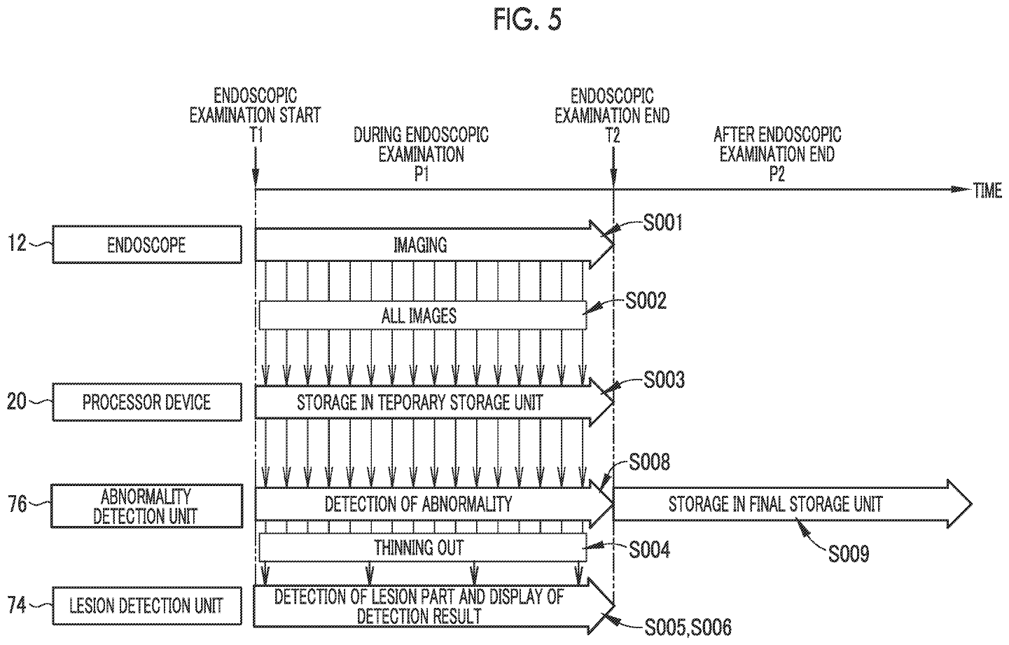

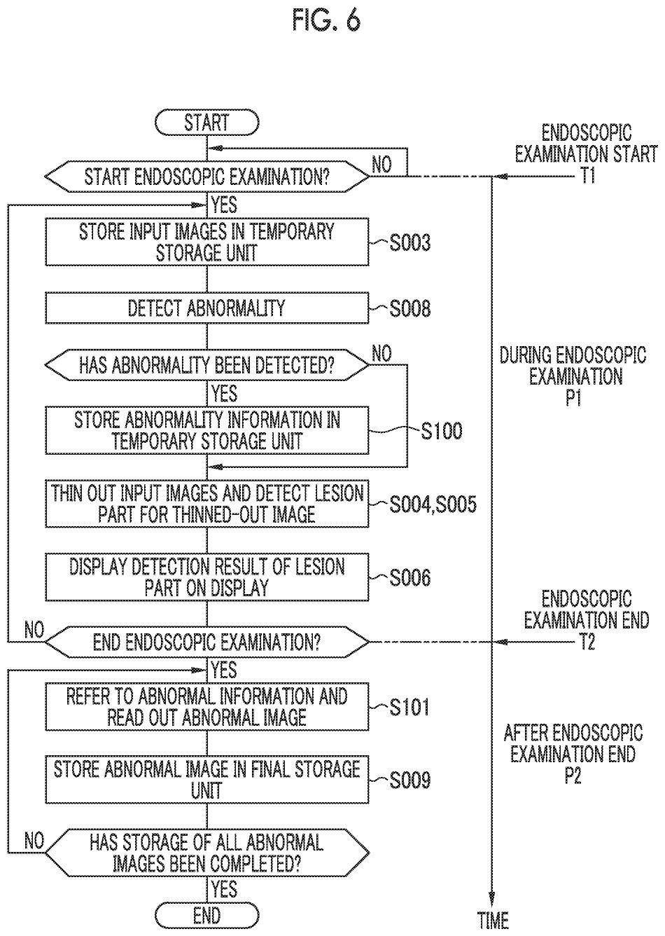

As shown in FIGS. 5 and 6, in the second embodiment, during the endoscopic examination (P1), the processor device 20 stores all the images input from the endoscope 12 in the temporary storage unit 62 (S003). In addition, the processor device 20 (abnormality detection unit 76) detects the abnormality for all the images input from the endoscope 12 (S008). Then, in a case where the abnormality is detected, the abnormality information is stored in the temporary storage unit 62 in association with the abnormal image (S100). As described above, the abnormality information is information regarding the detected abnormality (information indicating the type of the abnormality and the like), and may be stored as the accompanying information of the abnormal image in a form attached to the abnormal image, or may be stored separately from the abnormal image. In a case where the abnormality information is stored separately from the abnormal image, the correspondence information indicating the correspondence relationship between the abnormal image and the abnormality information need only be stored by being attached to the abnormal image or the abnormality information, or need only be stored independently.

Further, the processor device 20 (lesion detection unit 74) analyzes a thinned-out image obtained through thinning out (S004), which is a part of the images input from the endoscope 12, to detect the lesion part (S005) and displays the detection result on the display 16 (S006). The processing, specifically, the storage of the image in the temporary storage unit 62 (S003), the detection of the abnormality and the storage of abnormality information in the temporary storage unit 62 (S008 and S100), and the detection of the lesion part and the display of the detection result (S004 to S006) are repeated during the endoscopic examination (P1) (from the endoscopic examination start (T1) to the endoscopic examination end (T2).

With the endoscopic examination end (T2), that is, after the endoscopic examination end (P2), the processor device 20 refers to the abnormality information stored in the temporary storage unit 62 to read out the abnormal image associated with the abnormality information from the temporary storage unit 62 (S101), and stores the abnormal image in the final storage unit 64 (abnormal image storage area 68) (S009). Of course, the abnormality information in addition to the abnormal image may also be read out from the temporary storage unit 62 and stored in the final storage unit 64 (abnormal image storage area 68) in association with the abnormal image. As described above, in the second embodiment, a configuration is employed in which the storage of the abnormal image in the final storage unit 64 (abnormal image storage area 68) is performed after the endoscopic examination end (P2), so that the processing load during the endoscopic examination (P1) can be reduced.

In a case where the abnormality is detected during the endoscopic examination (P1) as in the second embodiment, it is preferable to exclude the abnormal image from the detection target of the lesion part. Specifically, the abnormality detection unit 76 executes the detection of the abnormality (S008) prior to the detection of the lesion part (S005) by the lesion detection unit 74 (the abnormality is detected before the lesion part is detected). Then, the lesion detection unit 74 detects the lesion part (S005) for a remaining image (hereinafter, referred to as a normal image) obtained by excluding the image in which the abnormality has been detected by the abnormality detection unit 76 (that is, the abnormal image) from the images input from the endoscope 12 as a target. By doing so, the lesion part can be more accurately detected. Of course, the lesion part may be detected on the thinned-out image obtained through thinning out, which is a part of the normal images.

In addition, in the above first and second embodiments, an example has been described in which the final storage unit 64 is provided integrally with the processor device 20 (incorporated into the processor device 20), but the present invention is not limited to this. The final storage unit 64 (one or both of the examination image storage area 66 and the abnormal image storage area 68) may be provided separately from the processor device 20 and may be connected to the processor device 20 via a network such as a LAN or the Internet.

The information stored in the final storage unit 64 includes personal information such as information regarding a subject person of the endoscopic examination (subject person information) and information regarding an operator of the endoscopic examination (operator information). For this reason, for access to the final storage unit 64, it is preferable to impose restrictions on the accessible range, such as allowing access only to a person (or a terminal) set in advance. Of course, different accessible ranges may be set between the examination image storage area 66 and the abnormal image storage area 68, such as allowing the examination image storage area 66 to be accessible only to a doctor or other personnel of a medical institution in which the endoscope 12 is disposed or allowing the abnormal image storage area 68 to be accessible only to repair personnel or other personnel of the manufacturer of the endoscope 12.

In addition, since the information stored in the examination image storage area 66 is also used for the endoscopic examination and the subsequent treatment, there is a high probability of needing to refer to the above-described personal information (the subject person information, the operator information, or the like) after the endoscopic examination. On the other hand, since the information stored in the abnormal image storage area 68 is used for the repair or maintenance of the endoscope 12, there is a low probability of needing to refer to the personal information after the endoscopic examination. Therefore, in a case where the information is stored in the abnormal image storage area 68, it is preferable to delete the personal information from the information to be stored.

Further, as described above, since the information stored in the examination image storage area 66 and the information stored in the abnormal image storage area 68 have different purposes for use, the functions required for managing the information, such as editing (displaying (playing back), moving, cutting (copying), pasting, deleting, and the like), are also different due to this difference. For this reason, it is preferable to differentiate the types of editing functions realized in managing the information and editing screens such as display menus for displaying the editing functions realized in managing the information between a case of managing the information stored in the examination image storage area 66 and a case of managing the information stored in the abnormal image storage area 68.

### Third Embodiment

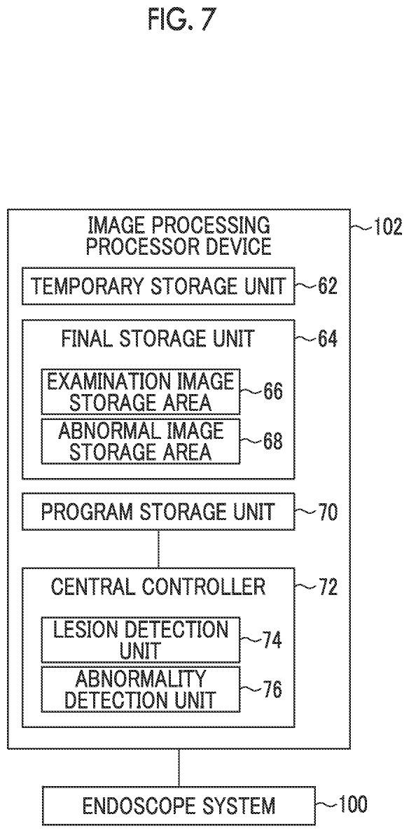

In the above first and second embodiments, an example has been described in which the processor device 20 constituting the endoscope system 10 functions as the processor of the embodiment of the present invention, but as shown in FIG. 7, in a third embodiment, an image processing processor device 102 functioning as the processor of the embodiment of the present invention is provided separately from an endoscope system 100.

The endoscope system 100 includes the endoscope 12 (see FIG. 1), the light source device 14 (see FIG. 1), the display 16 (see FIG. 1), and the like. The image processing processor device 102 is provided with the temporary storage unit 62, the final storage unit 64, the program storage unit 70, and the central controller 72. The program storage unit 70 stores a program related to various types of processing, control, or the like, and the central controller 72 functions as the lesion detection unit 74 and the abnormality detection unit 76 by operating the program stored in the program storage unit 70.

The image captured by the endoscope system 100 (endoscope 12) is input to the image processing processor device 102. The image processing processor device 102 detects the lesion part through the lesion detection unit 74 during the endoscopic examination (P1) using the input image, and inputs the result to the endoscope system 100. In the endoscope system 100, the detection result of the lesion part input from the image processing processor device 102 is displayed on the display 16. In addition, the image processing processor device 102 uses the image input from the endoscope system 100 (endoscope 12) to detect the abnormality during the endoscopic examination (P1) or after the endoscopic examination end (P2). Then, after the endoscopic examination end (P2), the abnormal image is stored in the final storage unit 64.

As described above, even in the third embodiment in which the processor (the image processing processor device 102) is provided separately from the endoscope system 100, the processing load during the endoscopic examination (P1) can be reduced similarly to the above-described first and second embodiments. In the present embodiment, an example has been described in which the image processing processor device 102 functions independently as the processor of the embodiment of the present invention, but a configuration may also be employed in which the image processing processor device 102 and the processor device 20 (see FIG. 1) of the endoscope system 100 function as the processor of the embodiment of the present invention in cooperation with each other.

In the above embodiments, as a hardware structure of the processing unit that executes various types of processing, such as the central controller 72, the lesion detection unit 74, and the abnormality detection unit 76, various processors are used as follows. The various processors include a central processing unit (CPU) that is a general-purpose processor which functions as various processing units by executing software (programs), a programmable logic device (PLD) that is a processor of which a circuit configuration is changeable after manufacturing, such as a field programmable gate array (FPGA), a dedicated electrical circuit that is a processor which has a circuit configuration exclusively designed to execute various types of processing, and the like.

One processing unit may be composed of one of these various processors or a combination of two or more of the processors of the same type or different types (for example, a plurality of FPGAs or a combination of a CPU and an FPGA). Alternatively, a plurality of processing units may be composed of one processor. A first example in which a plurality of processing units are composed of one processor is an aspect in which one or more CPUs and software are combined to constitute one processor and the processor functions as a plurality of processing units, as typified by a computer, such as a client or a server. A second example is an aspect in which a processor that realizes functions of an entire system including a plurality of processing units with one integrated circuit (IC) chip is used, as typified by a system on chip (SoC) or the like. As described above, various processing units are composed of one or more of the above various processors as hardware structures.

Furthermore, as the hardware structure of the various processors, more specifically, an electrical circuit (circuitry) in which circuit elements, such as semiconductor elements, are combined is used.

### EXPLANATION OF REFERENCES

10, 100: endoscope system
12: endoscope
14: light source device
16: display (monitor)
18: user interface
20: processor device (processor)
30: insertion part
32: operation part
34: bending portion
36: distal end portion
38: inlet
40, 42, 44: rotating dial
46, 48, 50: press button
60: image sensor
62: temporary storage unit (second storage device)
64: final storage unit (first storage device)
66: examination image storage area
68: abnormal image storage area
70: program storage unit
72: central controller
74: lesion detection unit
76: abnormality detection unit
102: image processing processor device

What is claimed is:

1. An endoscope system comprising:
an endoscope that images an inside of a body cavity; and
one or more processors configured to:
detect a lesion part from an endoscopic image captured by the endoscope, and display a detection result of the lesion part on a monitor during an endoscopic examination using the endoscope; and
detect an abnormality that has occurred in the endoscopic image, and store the endoscopic image in which the abnormality has been detected in a first storage device for storage after an end of the endoscopic examination.

2. The endoscope system according to claim 1,
wherein the one or more processors are configured to:
store the endoscopic image in a second storage device for temporary storage during the endoscopic examination, and detect the abnormality using the endoscopic image stored in the second storage device after the end of the endoscopic examination.

3. The endoscope system according to claim 1,
wherein the one or more processors are configured to:
detect the abnormality during the endoscopic examination.

4. The endoscope system according to claim 3,
wherein the one or more processors are configured to:
detect the abnormality before detecting the lesion part; and
exclude the endoscopic image in which the abnormality has been detected, from a detection target of the lesion part.

5. The endoscope system according to claim 1,
wherein the one or more processors are configured to:
detect the lesion part for a thinned-out endoscopic image which is a part of a plurality of the endoscopic images.

6. The endoscope system according to claim 5,
wherein the one or more processors are configured to:
store the endoscopic image in which the abnormality has been detected in the first storage device in a form accessible to an external device.

7. The endoscope system according to claim 6,
wherein the endoscopic image includes personal information including at least any of subject person information regarding a subject person of the endoscopic examination or operator information regarding an operator of the endoscopic examination, and
the one or more processors are configured to:
erase the personal information from the endoscopic image in which the abnormality has been detected and store the endoscopic image in the first storage device.

8. The endoscope system according to claim 6,
wherein the one or more processors are configured to:
set an allowable access range for the endoscopic image in which the abnormality has been detected to a range different from an allowable access range for an endoscopic image used for the endoscopic examination.

9. The endoscope system according to claim 6,
wherein the one or more processors are configured to:
set an allowable editing type for the endoscopic image in which the abnormality has been detected to a type different from an allowable editing type for an endoscopic image used for the endoscopic examination.

10. The endoscope system according to claim 6,
wherein the abnormality includes an abnormality based on an internal factor including a malfunction occurring in a main body of the endoscope and an abnormality based on an external factor occurring outside the main body of the endoscope, and
the one or more processors are configured to:
detect the abnormality based on the internal factor.

11. An operation method of an endoscope system, the method comprising:
a step of detecting a lesion part from an endoscopic image captured by an endoscope, and displaying a detection result of the lesion part on a monitor during an endoscopic examination using the endoscope; and
a step of detecting an abnormality that has occurred in the endoscopic image, and storing the endoscopic image in which the abnormality has been detected in a first storage device for storage after an end of the endoscopic examination.

12. One or more processors that are connected to an endoscope system including an endoscope which images an inside of a body cavity and that receives an input of an endoscopic image captured by the endoscope during an endoscopic examination using the endoscope, the one or more processors configured to:
during the endoscopic examination, detect a lesion part from the endoscopic image and input a detection result of the lesion part to the endoscope system; and
detect an abnormality that has occurred in the endoscopic image, and store the endoscopic image in which the abnormality has been detected in a first storage device after an end of the endoscopic examination.
